(19) United States
(12) Patent Application Publication
(10) Pub. No.: US 2024/0366116 A1
(43) Pub. Date: Nov. 7, 2024

(54) DEVICE AND METHOD USING RESPIRATORY INDUCTANCE PLETHYSMOGRAPHY

(71) Applicant: NETSENSING TECHNOLOGY SARL, Neuchatel (CH)

(72) Inventors: Raja Yazigi, La Neuveville (CH); Philippe Koller, Neuchatel (CH)

(21) Appl. No.: 18/292,789

(22) PCT Filed: Jul. 22, 2022
(86) PCT No.: PCT/IB2022/056799

§ 371 Date: Jan. 26, 2024

(30) Foreign Application Priority Data
Jul. 27, 2021 (EP) 21188022.4

(51) Int. Cl. A61B 5/113; A61B 5/00; A61B 5/08

(52) U.S. Cl. CPC A61B 5/1135; A61B 5/0803; A61B 5/0826; A61B 5/6831; A61B 2560/0462; A61B 2562/164; A61B 2562/227

(57) ABSTRACT

The present invention relates to a Respiratory Induction Plethysmography device comprising at least one folded wire extendable in a first direction (D1) and at least one second wire extendable in a second direction (D2), each of these wires being operated at different frequencies. The present invention further relates to disposable stretchable bands adapted therefor and to a method of RIP measurement, adapted for the identification of dorsal apnea.

### TECHNICAL DOMAIN

The present invention concerns a wearable device adapted for monitoring a patient during sleep. It relates in particular to a wearable device used for respiratory inductance plethysmography. It further relates to a method of monitoring the patient during sleep, and more particularly monitoring the sleep apnea syndrome.

### RELATED ART

Sleep apnea syndrome (SAS) is a sleep disorder in which an individual's breathing repeatedly reduces during sleep, sometimes tens or hundreds of times. One common form of SAS is the obstructive sleep apnea (OSA) where the pause in breathing is due to a blocked airway, usually when the soft tissue in the back of the throat collapses during sleep. Another form of SAS is central sleep apnea (CSA) where the pause is commanded from the brain. Another form of the SAS is hypopnea where respiration is reduced along with a oxygen desaturation or a micro wake up.

If untreated, SAS can result in a number of health problems, including heart failure such as sudden cardiac death. Sudden death is widespread both among newborns and among adults.

The use of Respiratory Inductance Plethysmography (RIP) is commonly used to this end. Stretchable belts comprising an electrically conductive wire are placed around the chest and around the abdomen of a patient to monitor both thoracic and abdominal breath. The resonance frequency of the plethysmography wire loop varies during the breath of the patient since the area of the loop varies. The variation of the inductance of the wire loops related to the two belts can be analysed to determine the apnea events of the patient during the sleep.

The wires have been object of extensive alternatives to improve the sensitivity of the measurement. The document US2004225227A1 discloses an elastic belt wherein the conductor has hairpin-like feature. The document U.S. Pat. No. 6,341,504B1 provide devices comprising multiple parallel wires. The documents US20110087115, U.S. Pat. Nos. 5,159,935 and 4,373,534 provide different plethysmography devices.

While the sensitivity has been a parameter of interest so far, several limitations remain with regard to the Respiratory Inductance Plethysmography. One of those resides in the fact that it can be difficult to distinguish the move related to the breath of the patient from the other movements that a patient can have during night. In addition, the known devices do not allow to determine the position of the patient. In particular, the known RIP belts only determine the area variation of the chest and abdomen of the patient without determining if the patient is lying on is back or on his face. It is thus necessary to use additional monitoring devices such as cameras or additional sensors such as 3D accelerometer, and to correlate the data of more than one device to determine if the apnea events occur when the patient his on his back or in another position. For example, the American Academy of Sleep Medicine (AASM) mentions that the dorsal apnea should be distinguish from the non-dorsal apnea. For example, the obstructive sleep apnea may be associated to a dorsal position of the patient, which may request specific treatment conditions.

Furthermore, the RIP belts currently used are disposable devices. They are normally used for a given patient, or for a predetermined period of monitoring. The RIP belts are designed to be combined with a non-disposable oscillators. They are thus certified to provide reliable measurements with such dedicated oscillators. However, using a type of belt with an oscillator not specifically dedicated to such a belt steel remains possible, which may provide erroneous measurements. In addition, there is no guarantee in such a situation that no significant variation of measurement occurs from one RIP belt to another one.

Another drawback of the current devices relates to the fact that the belts, worn by the user when sleeping, should be connected to an oscillator, arranged at distance from the user. The connexion cables thus generate discomfort to the user, who may feel embarrassed by the cables, which can disturb his sleeping quality in addition to his apnea.

### Short Disclosure of the Invention

An aim of the present invention is the provision of a wearable device adapted for Respiratory Inductance Plethysmography, that overcomes the shortcomings and limitations of the state of the art. It is in particular an aim to provide a wearable device which allows determining the dorsal position of a patient while detecting a sleep apnea event. It is an aim to limit the number of sensors when determining such a dorsal position. It is a further aim to limit the hindrance of the sensing devices so as to limit the discomfort of a patient.

It is a further aim of the present invention to provide a disposable wearable device which is adapted to a given oscillator. It is also aimed at preventing usage of non-authorized or non-valid disposable device.

Another aim of the invention is to provide a method for monitoring a patient during sleep. In particular, the present method aims at detecting and/or identifying sleep apnea syndrome and related sleep apnea indications.

According to the invention, these aims are attained by the object of the independent claims, and further detailed in the claims dependant thereon.

With respect to what is known in the art, the invention provides the advantage of a concomitant determination of an apnea event and the position of a patient, in particular the dorsal position of a patient, using a single detection device. More particularly, the single detection device is a RIP device. It further provides the advantage of a reliable measurement, since only authorized disposable stretchable bands are used for the measurements. Other advantages will appear through the following disclosure.

### SHORT DESCRIPTION OF THE DRAWINGS

Exemplar embodiments of the invention are disclosed in the description and illustrated by the drawings in which:

FIG. 1: Schematic representation of a device according to an embodiment of the present invention

FIG. 2: Schematic representation of a device according to another embodiment of the present invention

FIG. 3: Schematic representation of a connexion mean according to one embodiment of the present invention

FIG. 4: Schematic representation of a device and a connexion means according to an embodiment of the present invention

FIG. 5: Schematic representation of a device and a connexion means according to an embodiment of the present invention

FIG. 6: Schematic representation of a connexion mean according to one embodiment of the present invention

### EXAMPLES OF EMBODIMENTS OF THE PRESENT INVENTION

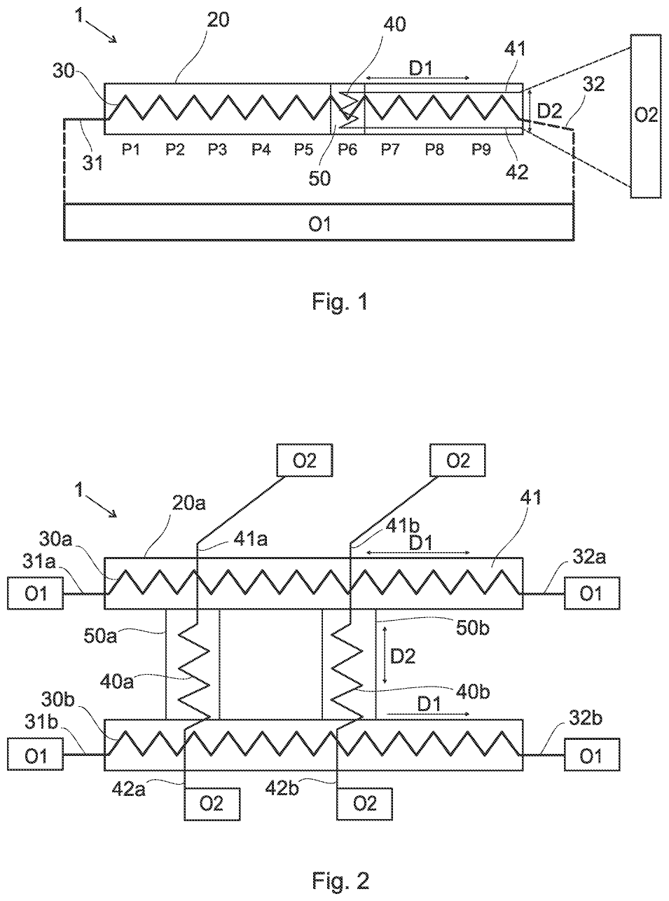

With reference to FIG. 1, the wearable device 1 comprises at least one stretchable band 20 the extremities of which can be removably joined by a first connexion mean. To this end, any mean of connexion of the two extremities may be used, such as scratch, snaps, boucle, hook, clamp and related means. According to a specific embodiment, the first connexion means is a connecting member 100, 100′ described below. The stretchable band 20 is stretchable along a first stretch direction D1. Preferably, the first stretch direction D1 corresponds to the longitudinal dimension of the band 20, meaning the direction between the two extremities of the band 20. The stretchable band 20 can be stretchable in the first stretch direction D1 on all its length. Alternatively, it can comprise stretchable portions in the first stretch direction D1, which alternate with non-stretchable portions.

The stretchable band 20 is combined with at least a first wire 30 which is electrically conductive, and which is oriented along the first stretch direction D1. The first wire 30 is combined with the stretchable band 20 so as to not be straight when the stretchable band 20 is at rest, meaning not stretched. The first wire 30 occupies a certain width of the stretchable band 20, between one side and the other one, either in a regular way or in a non-regular way. For example, the stretchable band 20 can have a width comprised between around 5 to 20 cm, or around 8 to 15 cm, such as about 10 cm. The length of the stretchable band 20 is typically comprised between around 80 cm to around 180 cm so as to be arranged around the thorax of a patient. Adjusting means may be included to better fit the stretchable band 20 to the patient. The first wire 30 is connected to the stretchable band 20 at specific points so that it is free to extend when the stretchable band 20 is stretched, while remaining combined with the stretchable band 20. The term “combined” means associated to, or linked to, or integrated with. The first wire 20 is thus folded along the first stretch direction D1. The first wire 30 can be regularly folded along the full length of the stretchable band 20. Alternatively, it can be folded only at portions of the stretchable band 20, in particular when the stretchable band 20 comprises stretchable and non-stretchable portions. In this case, the folded portions of the first wire 30 correspond to the stretchable portions of the band 20.

The first 31 and second 32 ends of the first wire 30 can be connected to an oscillator (not represented) adapted to pulse an electrical courant through the wire 30. Depending on the stretching status of the stretchable band 20, the inductance related to the first wire 30 varies and can be measured to determine the respiratory parameters. Among the measured parameters, one can cite the respiratory rate, the volume corresponding to each breath, the minute ventilation, the peak inspiratory flow, the fractional inspiratory time, the work of breathing, the peak and the mean of inspiratory and expiratory flow.

The stretchable band 20 can comprise more than one first wire 30 arranged along the first stretch direction D1. In case several first wires 30 are included or combined to the first stretchable band 20, they may be all identical or be different from each other, or arranged differently from each other. For example, they can be differently folded, or have different size, or comprise folded portion at different positions of the stretchable band 20, or combine several of these distinctions.

The stretchable band 20 can further comprise one or several second stretchable portions 50 which are stretchable in a second stretch direction D2. Preferably, the second stretch direction D2 is a direction orthogonal to the first stretch direction D1. The second stretch direction D2 preferably corresponds to the direction of the vertebral column of the patient. Each of the second stretchable portions 50 comprises at least a second wire 40, which is electrically conductive, and which is oriented along the second stretch direction D2.

Preferably, the stretchable band 20 is stretchable in the first stretch direction D1 and in the second stretch direction D2 along its full length. It comprises one or several second wires 40 oriented along the second stretch direction D2.

The second wire 40 comprises a first 41 and a second 42 ends, which can be connected to an oscillator adapted to pulse an electrical courant through the wire 30. The second wire 40 comprises at least a folded portion which can be extended when the stretchable band 20 is stretched along the second stretch direction D2. The folded portion may be limited to the width of the first stretchable band 20. It can have for example a length of around 5 to 10 cm. The inductive loop thus comprises said folded portion and the non-folded conductive portions of the second wire, wherein at least the folded portion, or only the folded portion, is oriented along the second stretch direction D2.

According to an embodiment, the first wire 30 and the second wire 40 are both combined to the same stretchable band 20. In this case, the stretchable band 20 is stretchable, at least partly in both the first stretch direction D1 and the second stretch direction D2. Alternatively, the first wire 30 is combined to the stretchable band 20 and the second wire 40 is combined to a different stretchable band, stretchable in the second stretch direction D2 and crossing the first stretchable band 20. In this last case, the length of the folded portion of the second wire 40 can be larger than the width of the first stretchable band 20.

The second wire 40, whether combined to the same stretchable band 20 as the first wire 30 or to a different one, may cross the first wire 30 at a predetermine position P along the length of the stretchable band 20. For example, the second wire 40 may cross the first wire 30 at a position close to one of the extremities of the stretchable band 20. Alternatively, it can cross the first wire 30 at a more central position P of the stretchable band 20. Such a predetermined position P can correspond to a portion of the body of the patient once the wearable device is placed on the patient. For example, a position P close to an extremity of the stretchable band 20 can correspond to a frontal part of the patient such as the chest or the abdomen, when the stretchable band 20 is locked on a frontal side of the patient. A more central position P, on the stretchable band 20, would thus correspond to a dorsal part of the patient. Other position P corresponding to a lateral part of the patient may also be predetermined so that a wire 40 can be arranged thereon.

The stretchable band 20 can comprise only one predetermined position P, wherein a second wire 40 is arranged, crossing the first wire 30. The predetermined position P can be arranged so that the second wire 40 is placed on a dorsal or a facial or a lateral position of the patient, once the wearable device is placed. Alternatively, the stretchable band 20 can comprise at least two predetermined positions P where a second wire 40 crosses the first wire 30, each of the predetermined position P corresponding to a dorsal or a facial or a lateral portion of the patient's body. For example, two predetermined position P may be arranged, wherein one corresponds to a dorsal part of the patient and the other one corresponds to the facial part of the patient. The overall arrangement may of course be easily adapted according to the needs.

According to an advantageous arrangement, the first wire 30 is combined to the stretchable band 20 as above described, and the second wire 40 is combined to a different stretchable band, crossing the first stretchable band 20. The second stretchable band, comprising the second wire 40 can thus cross the first stretchable band 20 at any position P, selected according to the needs. The second stretchable band can for example be placed at a position corresponding to the dorsal part or to the facial part of the patient. Several second bands can also be envisaged and freely placed at the desired positions P. The second wire 40 comprises at least one folded portion oriented along the second stretch direction D2 so that an elongation of the second stretchable band can be detected. The length of such a folded portion can be comprised between around 5 to 10 cm, which can be sufficient to detect an elongation of the second stretchable band. It can also be longer if necessary.

Although the first stretchable band 20 may be independent from one or several second stretchable bands, crossing it, attachment points may be arranged on the first stretchable band 20, and/or on the second crossing stretchable bands. Such attachment points can be of any suitable type such as snaps, snap button, hook, passer-by etc. according to a specific arrangement such an attachment point can be defined by a connecting element 100, 100′ described below. The attachment points may be localised on predetermined positions P1, P2, P3 . . . . P9 along the first stretchable band 20, so that the second stretchable bands can be arranged across the first stretching band 20 at predetermined and reproducible positions.

According to an embodiment, the folded length of the second wire 40 is fully contained in the width of the first stretchable band 20. This is for example the case when both the first 30 and second 40 wires are combined to the same first stretchable band 20. In that case, the position of each of the second wire corresponds to one of the predetermined position P1, P2, P3 . . . . P9 above described. According to another embodiment, the second stretchable band, comprising the second wire 40, can form a loop around the patient's body, passing above his shoulders like suspenders.

The wearable device 1 may thus comprise at least one first stretchable band 20 comprising a first wire 30 and at least one second wire 40 crossing the first wire 30, the second wire being combined with the first stretchable band 20 or with another stretchable band. Preferably, the wearable device 1 comprises a set of one or several first stretchable bands 20, each comprising at least one first wire 30 comprising folded portions arranged along the first stretch direction D1, and one or several second wire 40, being combined to said first stretchable band 20 or to independent one, and comprising at least one folded portion arranged along the second stretch direction D2.

FIG. 2 illustrates an alternative arrangement wherein two first stretchable bands 20a, 20b are arranged parallel to one another, each one comprising at least one first wire 30a, 30b, said arrangement further comprising one or more second stretchable bands 50a, 50b, each one comprising at least one second wire 40a, 40b. The first stretchable bands 20a, 20b are stretchable in the first stretch direction D1 and the second stretchable bands 50a, 50b are stretchable in the second stretch direction D2. The one or several second stretchable bands 50a, 50b can be non-removably linked to the two first stretchable bands 20a, 20b. Alternatively, the one or several second stretchable bands 50a, 50b can be removably linked to the two first stretchable bands 20a, 20b by mean of some attachment points such as snaps, snap button, hook or equivalent means. According to a specific embodiment, such attachment point is defined by a connecting member 100, 100′ described below. The extremities of each second stretchable band 50a, 50b, or some of them, can join the two parallel first stretchable bands 20a, 20b. Alternatively, the second stretchable bands 50a, 50b, or some of them, can cross one or both of the first stretchable band 20a, 20b. The second stretchable bands 50a, 50b can be arranged at predetermined positions P1, P2, P3 . . . . P9. Some of predetermined positions correspond to a dorsal, a facial or a lateral position of the patient. Each of the second stretchable bands 50a, 50b is combined to at least one second wire, having at least one folded portion oriented along the second stretch direction D2. The folded portion of the second wires 40a, 40b may extend through the space between the two parallel first stretchable bands 20. In this specific arrangement, the first stretchable band 20 may be stretchable in both D1 and D2 directions or only along the first stretch direction D1.

Independently of the special arrangement of the stretchable bands, both ends of each of the wires are connected to an oscillator. Preferably, the wearable device 1 comprises or is connected to two different oscillators. The first wire or wires of the first stretchable band 20, or the first multiple stretchable bands 20a, 20b when applicable, are connected to a first oscillator O1. The second wire or wires 40 are connected to a second oscillator O2. This is applicable when the second wire is combine to the same stretchable band as the first wire or to different stretchable bands. To this end, the wires comprise the necessary electrical connexion means adapted to be electrically connected to the related oscillator. Preferably, the first oscillator O1 works at a predetermine first frequency F1 and the second oscillator O2 works at a predetermine second frequency F2. Although the first F1 and the second F2 frequencies can be identical, they are preferably different from each other. For example, the frequencies F2 related to the second wires 40 may be 2, or 5 or 10 or 20 fold higher than the frequencies related to the first wires 30. The frequencies related to the second wires 40 may alternatively be lower than those related to the first wires 30, by the same ratios. Other ratios may be envisaged according to the needs. The range of applicable frequencies is suitable for the RIP measurements. It can be for example comprised between few Hz, such as around 10 Hz, and few tenth of KHz such as around 10 KHz or around 50 KHz or more.

When the wearable device 1 comprises two parallel first stretchable bands 20a, 20b, one of these two bands surrounds the chest of the patient and the other one surrounds the abdomen of the patient. The distance between the two stretchable bands 20a, 20b can be predetermined to this extend. Alternatively, the distance between the two parallel first stretchable bands 20a, 20b can be adapted. For example, when one or more second stretchable bands 50a, 50b are arranged in between, their length can be object of an adjustment so as to optimise the relative position of the two parallel first stretchable band 20a, 20b.

Whether the second wire 40 is comprised to the same first stretchable band 20 than the first wire or in an independent stretchable band, it is preferably arranged at a position corresponding the back of the patient.

The device here described also comprises a central unit adapted to measure the inductance and/or the variation of inductance. The first stretchable band 20 or assembly of first stretchable bands 20a, 20b, can be connected to, or comprise, a first central unit adapted to determine the inductance or the variation of inductance related to the first wires 30. The second stretchable band or assembly of second stretchable bands 50a, 50b, can be connected to, or comprise, a second central unit adapted to determine the inductance or the variation of inductance related to the second wires 40. The first central unit and the second central unit may be arranged as a single device or as two distinct devices as requested by the needs. A central unit according to the present disclosure can comprise or be connected to computational means adapted to identify an apnea event out of the inductance variations of the first wire or first set of wires 30, 30a, 30b. The computational means are further adapted to determine a position of the patient out of the inductance variation of the second wire or second set of wires 40, 40a, 40b. A central unit according to the present disclosure can be programmable with algorithms allowing to associate a position of the patient to an apnea event. In particular a dorsal position of the patient can be associated to an apnea event. A central unit can further be programmable with a specific position P of the second wire or with specific positions P1, P2, P3 . . . . P9 of the set of second wires, so that any position of the patient can be determined when an apnea event is detected. Such a central unit can be arranged remote the patient and receive the signal from suitable sensors placed on or near the stretchable bands. Alternatively, the central unit as well as the necessary computational means are arranged on the patient. According to a specific embodiment, a central unit corresponds to a connecting member 100, 100′ described below.

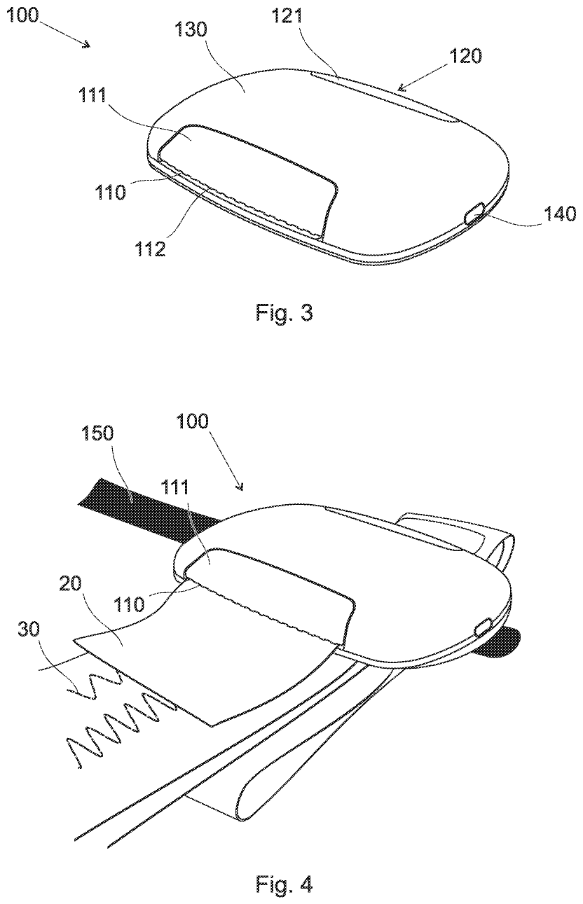

To be able to provide accurate measurements, the stretchable bands should have a correct length, corresponding to the size of the body of the patient. In particular, the ends of the stretchable bands should be properly joined and connected to an oscillator, which requires some manipulation. So as to simplify the positioning of the stretchable bands, the present wearable device 1 can comprise a connecting member 100, better shown in FIGS. 3 to 6. The connecting member 100 allows to clip the two extremities of a stretchable band so as to maintain it on the body of a patient. The connecting member 100 can comprise a casing 130 having two opposite slots 110, 120 in which a stretchable band can slide. The connecting member 100 comprises at least one clamping member 111, 121 adapted to clip a stretchable band once it has been inserted in a slot. The length of the slots of the connecting member 100 corresponds to the width of a given stretchable band so as to allow it sliding across the connecting member 100. The length of the slots 110, 121 can be determined so as to limit the usage of stretchable bands having the dedicated width. Both ends of a given stretchable band 20 can be inserted in the opposite slots 111, 121 of the connecting member 100 and clamped by mean of a clamping member 111, 121. Such a clamping member can take the form of a hinged blade movable between an open position and a clamping position. The open position allows the stretchable band sliding through the corresponding slot 110, 120. In its clamping position the clamping member is in close contact with the stretchable band so as to maintain it. Using such a connecting member 100, there is no need to cut the stretchable band at the desired length or to select a stretchable band of a suitable length. A stretchable band can slide from a first slot 110 until the opposite slot 121 and protrude outside the connecting member 100 so that the end can remain free, while the band is still maintained by the clamping member.

The clamping member 111, 121 may be provided with teeth 112 or other gripping means for better maintaining the stretchable bands. In addition, the slots can be provided with an electrical connector 113 adapted to be in contact with the wire or the set of wires associated to the stretchable band. Such an electrical connector 113 can take the form of a metallic rode arranged across the slot. By this way the wire is maintained in close contact to such an electrical connector once the corresponding stretchable band is clamped. Thus, the electrical connexion is established at the same time that the stretchable band is placed on the body. The electrical connector 113 can be linked to one or several inductance sensors adapted to determine the variation of inductance of a wire or a set of wires associated with the clamped stretchable band.

According to an embodiment, the connexion member 100 comprises an internal circuitry connecting the electrical connectors 113 of each slot 110, 120 and the corresponding inductance sensors to an external central unit.

According to a preferred embodiment, the connecting member 100 does not need to be connected to an external central unit. The casing 130 comprises an internal battery 160, the computing means above described 170 as well as the sensing elements adapted to measure the inductance variation of a stretchable band once clamped to the connecting member 100 (FIGS. 6 and 7). The connecting member 100 can further be provided with an activation member 140 such as a button, adapted to activate and deactivate the measurements. The connecting member 100 can further comprise recording means, such as memories and/or communication means adapted to record and communicate some data, and in particular the measured data. According to such an arrangement, the connecting member comprises a central unit. In case the second wire or set of wire 40a, 40b are included in the first stretchable band 20, the connecting member 100, 100′ can comprise specific electrical connexion allowing to sense the inductance of such second wire or set of wires. In addition, the position P of each one of the second wire 40a, 40b can be determined. For example, each one of the second wire can have a dedicated electrical connexion so as to be identified by the central unit. Under such an arrangement, the position of the patient can be determined by determining the second wire the inductance of which varies.

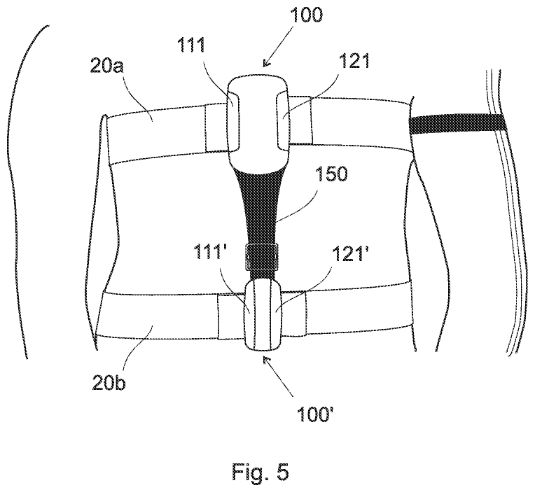

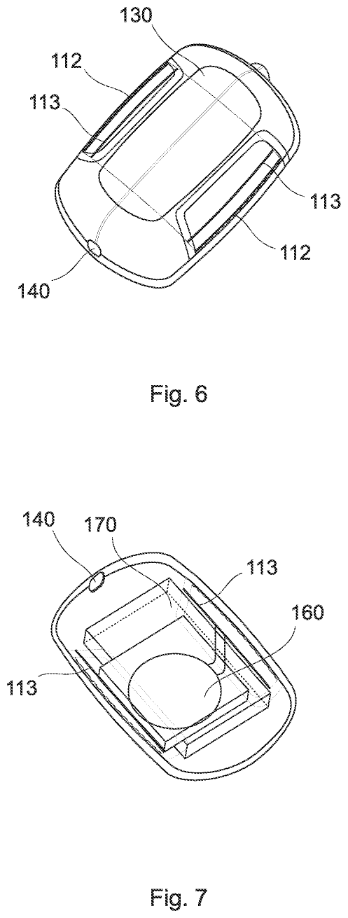

According to an embodiment better shown in FIG. 5, each stretchable band can be positioned with such a connecting member 100, 100′. In particular, when the wearable device 1 comprises a stretchable band 20a on chest and a stretchable band 20b on the abdomen, a first connecting member 100 maintains the first stretchable band 20a and a second connecting member 100′, having adapted slots and clamping members 111′, 121′, maintains the second stretchable bands 20b.

The size of the first 100 and second 100′ connecting members can be identical. Alternatively, the size of the first 100 and the second 100′ connecting members can be different from each other, and in particular the length of the corresponding slots. For example, larger stretchable bands 20a can be placed on chest while stretchable bands 20b having a lower width are placed on the abdomen or vice versa.

All of the connecting members 100, 100′ can comprise all the necessary circuitry to independently measure the inductance variation of the corresponding stretchable band and store or transmit the data. According to another embodiment, when several connecting members 100, 100′ are present, only one serves as a central unit and the other ones are only used as clamping elements to maintain the corresponding stretchable bands, and eventually also to sense the corresponding inductance. In this case, the secondary connecting members 100′ can be connected to a central connecting member 100 comprising all the necessary battery 160, computing means 170, inductance sensor, memory etc.

According to another embodiment, several connecting members 100, 100′ can be associated to a support 150 such as a belt. By this way, the distance between two connecting members 100, 100′ can be adjusted so as to maintain the two corresponding stretchable bands 20a, 20b at a proper distance from each other. When applicable, such a support can comprise the necessary electrical connections allowing to connect a connecting member 100′ to a central connecting member 100 acting as a central unit.

According to another embodiment, not shown, the connecting member can comprise more than 2 slots so as to receive more than one stretchable band. For example, a connecting member 100 can comprise a first pair of opposite slots as mentioned above, adapted to clamp a first stretchable band, and a second pair of opposite slots, arranged orthogonally to the first pair of opposite slots, so as to receive a second stretchable band crossing the first stretchable band. Such a connecting member comprise two distinct sensors each one being dedicated to one stretchable band. More particularly, the first pair of slots allows to clamp and connect the first wire or set of sires 30a, 30b. The second pair of slots allows to clamp and connect the second wires or set of wires 40a, 40b.

Such a connecting member 100 can define one of a predetermined position P above described and act as an attachment point above described, adapted to join a first stretchable band 20a, 20b with a second stretchable band 50a, 50b.

Although stretchable bands are here described, the device 1 also comprises garment or textiles or any related clothes comprising such stretchable bands, or being stretchable in such extend to correspond to the present disclosure.

The present disclosure also relates to a method for identifying sleep apnea syndrome using respiratory inductance plethysmography. It relates in particular to a method for determining the dorsal sleep apnea, using a combination of at least a first folded wire 30 and at least a second folded wire 40 which are stretchable in two different stretch directions D1 and D2. The first wire or wires are arranged so that they can extent around the chest and/or the abdomen of the patient. The inductance variation of the first wire or the first wires allow to monitor the breath of the patient. The second wire or the second wires 40 are arranged to cover the back side and/or the front side of the patient so as to identify extension on one of the back side or front side of the patient. The inductance variation of the second wire or second wires allow to determine the position of the patient. For example, when inductance varies on the second wires arranged on the facial side of the patient, it can be determined that the patient is sleeping on his back. On the contrary, when an inductance varies on the wire related to the back side of the patient, one can deduce that the patient sleeps on his facial side.

The second wire or second wires 40 may comprise a folded portion and a non-folded portion, so as to better localize the variation of the inductance. For example, a folded portion of a second wire 40 may be arranged either on the back side or on the facial side, and the non-folded portion may be arranged on the opposite side. By this way, a dorsal apnea can be identified. For example, the sensed data related to the first wire or to the first wires 30 can be combined to the sensed data related to the second wire or wires 40 so that the position of the patient is determined when an apnea event is detected. According to another possible arrangement, a given second stretchable band may comprises several different second wires 40, each comprising a folded portion, wherein the folded portion of one of these is arranged on a dorsal position and the folded portion of another wire is arranged on a facial position.

The method of the present disclosure comprises for instance a step a) of monitoring the breath of a patient by respiratory inductance plethysmography, using at least one first stretchable band 20, 20a, 20b and a first oscillator O1 working at a first frequency F1. The first stretchable band comprises at least one folded wire 30 and is arranged in the first stretch direction D1.

The method comprises a step b) of determining the position of the patient by respiratory inductance plethysmography, using at least a second electrically conductive wire 40, arranged in a second stretch direction D2 crossing the first direction D1. In particular, step b) allows to determine if the patient is sleeping on his back or not. Alternatively, step b) allows to determine whether the patient is sleeping on his back or on his facial side, or one of his lateral side. The position of the patient is determined based on the position of the second electrically conductive wire 40, the inductance of which varies. The position of the second electrically conductive wire 40 correspond to the predetermined positions P1, P2, . . . . P9 of the first stretchable band 20, 20a, 20b. For example, in case the inductance of a second wire positioned at back of the patient varies, or if it varies more or less than the inductance of the second wires placed at other P positions, it is determined that the patient is sleeping on his back. When considering the inductance variation of the second wires, an algorithm can be used to weight the inductance of each one of the second wires 40a, 40b so as to determine the position of the patient.

The method further comprises a step c) of computing the data related to the first wire or wires 30 and the data related to the second wire or wires 40 for a given period of time so as to determine the position of the patient for an identified apnea event. It is for example determined if the patient is sleeping on his back or not, or whether for an identified apnea event, the patient is sleeping on his back, or on his facial side or on one of his lateral sides.

The method comprises a step d) of identifying a dorsal apnea, based on the previous steps. Such dorsal apnea is revealed when the patient is considered as sleeping on his back at a moment an apnea event or a succession of apnea events is detected.

The method may comprise a further step of calibrating the first wire or wires 30 and/or the second wire or wires 40. The method also comprises a step of recognizing that the stretchable band 20 or the assembly of stretchable bands involved in the RIP measurement corresponds to reliable and authorized devices. A dedicated double measurement can be initiated to determine if the stretchable band 20 or the assembly of stretchable bands comprises at least one first wire 30 and at least on second wire 40, and if the first wire comprises a folded portion extendable along the first stretch direction D1 and the at least one second wire comprises a folded portion extendable along the second stretch direct D2. In case of absence of one or both of these first 30 and second 40 wires, or in case of damage thereof, the present device can provide an alert and/or stop or prevent the measurement. Additional control elements may be included to one or more of the above described stretchable bands. For example, RFID tag or equivalent means can be envisaged to follow the usage and/or the quality of the stretchable bands.

The present disclosure further relates to a disposable stretchable band 20 or an assembly of several disposable stretchable bands 20a, 20b, 50a, 50b arranged as above described. Such a disposable stretchable band 20 or assembly of several disposable stretchable bands 20a, 20b, 50a, 50b are preferably provided with two distinct electrical connexion means, one being adapted to connect a first wire 30 or a first assembly of wires 30 to a first oscillator O1 and a second one adapted to connect a second wire 40 or a second assembly of wires 40 to a second oscillator O2. Alternatively, the disposable stretchable bands of the present invention are free of connexion means. They are in addition of a unique length so that they can be adapted to any body. Such disposable bands are usable with a connecting member 100, 100′ as above described, and can be clamped at a proper position.

The present disclosure further covers a connecting member 100, 100′ as above described.

1. A wearable device adapted to be worn by a patient, comprising
at least one first stretchable band comprising at least one folded first electrically conductive wire having a first end and a second end, both of its first end and second end being connected to a first oscillator working at a first frequency, wherein said at least one first stretchable band is stretchable in a first stretch direction, wherein both extremities of said at least one stretchable band are joined together by means of a first connexion mean,
at least a second folded electrically conductive wire having a first end and a second end, both of its first end and second end being connected to a second oscillator working at a second frequency, different from the first frequency, wherein said second folded wire is adapted to be extended in a second stretch direction different from the first stretch direction,
wherein said at least one first electrically conductive wire and said at least second electrically conductive wire are connected to at least one central unit adapted to determine the corresponding inductance and/or variation of inductance,
wherein said at least one first stretchable band comprises predetermined positions along its length, and wherein said at least second conductive wire crosses said first stretchable band at said predetermined positions and wherein said central unit comprises computing means adapted to identify an apnea event out of the inductance variations of said at least one folded first electrically conductive wire and to determine a position of the patient out of the inductance variation of said at least a second folded electrically conductive wire.

2. Wearable device according to claim 1, wherein at least one of said predetermined positions corresponds to a dorsal position of the patient.

3. Wearable device according to claim 1, wherein said at least one second wire, or some of said at least one second wire, are independent from said first stretchable band and combined to at least one second stretchable band stretchable in said second stretch direction, wherein said at least one second stretchable band crosses said at least first stretchable band at one of said predetermined position by means of an attachment points.

4. Wearable device according to claim 1, comprising two first stretchable bands arranged parallel to each other and one or more second stretchable bands being arranged orthogonally to said first stretchable band, wherein at least on of said second stretchable bands is arranged with regards to said first stretchable bands at a position corresponding to the dorsal side of the patient.

5. Wearable device according to claim 1, wherein said at least one second wire, or some of said at least one second wire, are combined to said first stretchable band extending within the width of said first stretchable band so as to cross the at least one first wire at said predetermined position.

6. Wearable device according to claim 1, wherein said first connection means joining both extremities of said first stretchable band is a connecting member comprising a casing having a first pair of two opposite slots adapted to receive said first stretchable band and at least one clamping member adapted to maintain said first stretchable band.

7. Wearable device according to claim 6, further comprising an electrical connector adapted to contact said at least one folded first electrically conductive wire once the corresponding stretchable band is clamped, and wherein said central unit is included in said connecting member.

8. Wearable device according to claim 6, wherein said connecting member further comprises a second pair of slots arranged orthogonal to the first pair of slots so as to receive a second stretchable band orthogonal to the first stretchable band, wherein said connecting member corresponds to an attachment point at said predetermined positions.

9-12. (canceled)

13. A method of determining the position of a patient while monitoring the breath of the patient by means of the wearable device according to claim 1, comprising:
a step a) of monitoring the breath of a patient by respiratory inductance plethysmography, using at least one first stretchable band combined to at least one first folded wire and a first oscillator working at a first frequency, said first stretchable band being stretched in a first stretch direction;
a step b) of determining the position of the patient by respiratory inductance plethysmography, using at least a second folded electrically conductive wire, arranged in a second stretch direction crossing the first direction and connected to a second oscillator working at a second frequency, different from said first frequency, wherein said at least one second folded electrically conductive wire is placed at a predetermined position along the first stretchable band,
a step c) of computing the data related to said at least one first wire and the data related to said at least one second wire for a given period of time, and
a step d) of determining a position of a patient during an apnea event, based on the computed data.

14. Method according to claim 14, further comprising a step of checking the presence and the integrity of both the at least one first wire and the at least one second wire.

15. Wearable device according to claim 1, wherein said central unit is programmable with algorithm allowing to associate a position of the patient to an apnea event.

16. Wearable device according to claim 1, wherein said central unit is programmable with specific predetermined positions of said at least second conductive wire, so that any position of the patient can be determined when an apnea event is detected.

17. Wearable device according to claim 6, wherein said connecting member corresponds to one of said at least one central unit.

18. Wearable device according to claim 6, wherein said connecting member further comprises recording means and/or communication means adapted to record and/or communicate sensed data.

19. Wearable device according to claim 6, wherein said connecting member comprises a first connecting member maintaining a first stretchable band and a second connecting member maintaining a second stretchable band, said first and second connecting members being associated to a support, so as to maintain the first and second stretchable bands at a predetermined distance from each other.
